(19) United States
(12) Patent Application Publication
(10) Pub. No.: US 2010/0151469 A1
(43) Pub. Date: Jun. 17, 2010

(54) SAMPLE-PROCESSING REAGENT COMPOSITIONS, METHODS, AND DEVICES

(76) Inventors: Wensheng Xia, Woodbury, MN (US); Ranjani V. Parthasarathy, Woodbury, MN (US)

Correspondence Address: 3M INNOVATIVE PROPERTIES COMPANY, PO BOX 33427, ST. PAUL, MN, 55133-3427, US

(21) Appl. No.: 12/597,394

(22) PCT Filed: Apr. 25, 2008
(86) PCT No.: PCT/US08/61488

§ 371 Date: Oct. 23, 2009

Related U.S. Application Data
(60) Provisional application No. 60/913,812, filed on Apr. 25, 2007.
(60) Provisional application No. 60/913,813, filed on Apr. 25, 2007.
(60) Provisional application No. 60/913,814, filed on Apr. 25, 2007.

(51) Int. Cl. C12Q 1/68; C12N 9/00; C12N 9/48; C12N 9/36; C12N 9/50; C12N 9/42; C12N 1/06; C40B 40/10; C12M 1/00

(52) U.S. Cl. 435/6; 435/183; 435/212; 435/206; 435/219; 435/209; 435/259; 506/18; 435/306.1

(57) ABSTRACT

Stable compositions comprising a nucleic acid binding support material comprising a substrate with functional groups attached to the substrate; a lysing enzyme; a water dispersible matrix material; and a saccharide, methods of using the compositions, and products and devices which include the compositions are disclosed.

This application claims the benefit of U.S. Provisional Application Nos. 60/913,812, filed Apr. 25, 2007; 60/913,813, filed Apr. 25, 2007; and 60/913,814, filed Apr. 25, 2007, each of which is incorporated herein by reference in its entirety.

### BACKGROUND

Isolating a biological material, for example, cells, viruses, and polynucleotides, from a sample can be useful when applying methods for detecting or assaying the biological material. Such methods often involve the identification of a polynucleotide or a portion of a polynucleotide for diagnosing a microbial infection, detecting genetic variations, typing tissue, and so on. Methods for identifying polynucleotides include, for example, amplifying, hybridizing to a known probe, and sequencing the polynucleotide. Amplifying methods include, among a number of others, polymerase chain reaction (PCR) and transcription-mediated amplification (TMA). Separating polynucleotides from a sample, which is often a complex mixture, can be necessary because large amounts of cellular or other contaminating material such as carbohydrates and proteins can interfere with these methods.

Methods are known for isolating polynucleotides from a sample. Some of these involve a time consuming series of extraction and washing steps. For example, nucleic acids have been isolated from a sample, such as a blood sample or a tissue sample, by lysis of the biological material using a detergent or chaotrope, extractions with organic solvents, precipitation with ethanol, centrifugations, and dialysis of the nucleic acid.

Solid extraction has also been employed in certain methods of isolating nucleic acids. Here the uses of particles, including microbeads, and membrane filters have been practiced. For example, DNA extraction has been carried out by absorption of DNA onto silica particles under chaotropic conditions. However, a subsequent washing step typically requires an organic solvent such as ethanol or isopropanol. Other examples of such methods have been reported, which include utilizing a hydrophobic surface in the presence of certain surfactants or polyethylene glycol, together with a high concentration of a salt. The use of organic solvents or high concentrations of salt limits the versatility of the extraction method for combining with subsequent methods such as nucleic acid amplification in microfluidic systems. Moreover, the use of multiple reagents during the extraction process is costly and time consuming. In another example, ammonium groups bound to a surface are used to attract and bind DNA molecules. DNA extraction kits having this capability are available, for example, from Qiagen (Valencia, Calif.). Eluting the adsorbed DNA is normally done at high pH or high concentration of salt, which can interfere with subsequent methods such as DNA amplification. Significant dilutions of the acquired material which can result in reduced sensitivity, or de-salting, or neutralization may be required.

An immobilized metal affinity chromatography (IMAC) method for separating and/or purifying compounds containing a non-shielded purine or pyrimidine moiety or group, such as nucleic acid, has been reported (U.S. Publication No. 2004/0152076A1). However, double stranded DNA was found not to bind to the column matrix.

Cell isolation and nucleic acid purification have been integrated by using the same solid phase for both cell adsorption and nucleic acid purification (U.S. Pat. No. 6,617,105). Here cells are bound to a solid support and isolated from the sample. The isolated cells are then lysed, and nucleic acids released from the cells are precipitated onto the same solid support.

With the large amount of interest in this area, there is a continuing need for materials and methods which are simple enough to extract polynucleotides under mild conditions and sufficiently versatile to be used with subsequent methods without interfering with such methods, or which may provide value by reducing labor.

### SUMMARY OF THE INVENTION

It has now been found that certain nucleic acid binding support materials comprising a substrate with functional groups attached to the substrate can be combined with lysis enzymes to provide a convenient and stable combination of reagents in a composition for processing a sample material. In certain embodiments, where the composition includes a liquid carrier, the composition has been found to be reproducibly printable to provide controlled amounts of lysis enzyme, support material, and other useful components. In certain embodiments, where the composition is in dry form, the composition has been found to be stable over extended periods of time, without degradation of activity or function. The composition can be used to simultaneously lyse microorganisms and capture the polynucleotides released from the microorganisms.

Accordingly, in one embodiment, the present invention provides a composition for processing a sample material, the composition comprising:

a nucleic acid binding support material comprising a substrate with functional groups attached to the substrate;

a lysing enzyme;

a water dispersible matrix material; and

a saccharide selected from the group consisting of a monosaccharide, an oligosaccharide, and a combination thereof.

In another embodiment, there is provided a method of processing a sample material comprising:

providing a composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof;

providing a sample material suspected of having a plurality of microorganisms which can be lysed; and

contacting the sample material with the composition;

wherein as least a portion of the microorganisms are lysed, the lysed microorganisms release nucleic acids, and at least a portion of the released nucleic acids are captured by the support material.

In another embodiment, there is provided a product comprising an array of spots on a carrier sheet, wherein at least a portion of the spots comprises a composition for processing a sample material, the composition comprising:

a nucleic acid binding support material comprising a substrate with functional groups attached to the substrate;

a lysing enzyme;

a water dispersible matrix material; and

a saccharide selected from the group consisting of a monosaccharide, an oligosaccharide, and a combination thereof.

In another embodiment, there is provided a device for process a sample material, the device comprising:

at least one process chamber defining a volume for containing the sample material or a portion thereof; and

a composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof;

wherein the composition is in the at least one process chamber.

### DEFINITIONS

The term “assaying” includes identifying a polynucleotide and/or determining the quantity of a polynucleotide that is present in a sample material.

As used herein the term “polynucleotide” refers to single and double stranded nucleic acids, oligonucleotides, compounds wherein a portion of the compound comprises an oligonucleotide or polynucleotide, and peptide nucleic acids (PNA), and includes linear and circular forms. For certain embodiments, the polynucleotide is preferably a single or double stranded nucleic acid.

As used herein, the term “substrate” refers to a material with a solid surface, which remains intact when contacted with an aqueous liquid. Some examples include a gel, a film, a membrane, a strip, a fiber, a plurality of particles, an interior wall of a column, tube, well, or container, a combination thereof, and the like.

“Microfluidic device” refers to a device with one or more fluid passages, chambers, or conduits that have at least one internal cross-sectional dimension, e.g., depth, width, length, diameter, etc., that is less than 500 μm, and typically between 0.1 μm and 500 μm. In the devices used in the present invention, the microscale channels or chambers preferably have at least one cross-sectional dimension between 0.1 μm and 200 μm, more preferably between 0.1 μm and 100 μm, and often between 1 μm and 20 μm. Typically, a microfluidic device includes a plurality of process chambers (e.g., chambers for mixing, separating, waste containment, diluting reagent, amplification reaction, sample or reagent loading, and the like), each of the chambers defining a volume for containing a sample; and at least one distribution channel connecting the plurality of chambers; wherein at least one of the chambers includes a composition described herein.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Also herein, the recitations of numerical ranges by endpoints include all numbers subsumed within that range (e.g., a pH of 7 to 10 includes a pH of 7, 7.5, 8.0, 8.7, 9.3, 10, etc.).

The above summary of the present invention is not intended to describe each disclosed embodiment or every implementation of the present invention. The description that follows more particularly exemplifies illustrative embodiments.

### BRIEF DESCRIPTIONS OF THE FIGURE

FIG. 1 is a top view of a device according to the present invention with two separate chambers and with the composition for processing a sample material in one of the chambers.

### DETAILED DESCRIPTION OF ILLUSTRATIVE EMBODIMENTS OF THE INVENTION

The present invention provides compositions, methods, products, and devices, that can be used for isolating a polynucleotide from a sample material by simultaneously lysing microorganisms from the sample material and capturing the polynucleotides therefrom. It has been found that lysis enzymes do not interfere with the polynucleotide binding to the support material, nor does the support material with functional groups destroy activity of the lysis enzymes during storage or use. Optionally, the isolated polynucleotides can be assayed, labeled, processed, or a combination thereof.

In one embodiment, there is provided a composition for processing a sample material, the composition comprising: a nucleic acid binding support material comprising a substrate with functional groups attached to the substrate; a lysing enzyme; a water dispersible matrix material; and a saccharide selected from the group consisting of a monosaccharide, an oligosaccharide, and a combination thereof.

In another embodiment, there is provided a method of processing a sample material comprising: providing a composition comprising a nucleic acid binding support material comprising a substrate with functional groups attached to the substrate, a lysing enzyme, a water dispersible matrix material, and a saccharide selected from the group consisting of a monosaccharide, an oligosaccharide, and a combination thereof; providing a sample material suspected of having a plurality of microorganisms which can be lysed; and contacting the sample material with the composition; wherein as least a portion of the microorganisms are lysed, the lysed microorganisms release nucleic acids, and at least a portion of the released nucleic acids are captured by the support material.

For certain embodiments, the above method further comprises separating the immobilized-metal support material with captured nucleic acids from any remaining materials. This can be carried out by decanting, pipetting, flushing, forcing the remaining material out of a process chamber using a pressure differential or a g-force, or otherwise removing the remaining materials from the support material.

For example, when the substrate is a wall of a container, the remaining material would be the material held by the container except for that which is captured by the support material and bound to the wall of the container. In another example, when the substrate is particles, the remaining material would be all material except for the particles and material bound thereto. In this example, techniques such as centrifugation and magnetic compaction of the particles may be used prior to removal of the remaining material. Separating the immobilized-metal support material with captured nucleic acids from any remaining materials may also include one or more washings. For certain embodiments, an aqueous buffer solution at a pH of 4.5 to 9 or 4.5 to 6.5 may be used. Examples of wash buffers include, for example, MES buffer, Tris buffer, HEPES buffer, phosphate buffer, TAPS buffer, and DIPSO (3-(N,N-bis[2-hydroxyethyl]amino)-2-hydroxypropanesulfonic acid) buffer. The support material could be packed as a flush column. The bound nucleic acids can be cleaned by using wash buffers to remove the unwanted species.

For certain embodiments, including any one of the above embodiments of the method, the method further comprises releasing the captured nucleic acids from the immobilized-metal support material. Releasing or eluting the captured nucleic acids can be carried out using an elution reagent. Examples of a suitable elution reagent include TES buffer, DIPSO buffer, TEA buffer, Tris buffer, phosphate buffer, pyrophosphate buffer, HEPES buffer, POPSO buffer, tricine buffer, bicine buffer, TAPS buffer, ammonium hydroxide, and sodium hydroxide. For certain embodiments, the releasing is carried out with an elution reagent selected from the group consisting of a phosphate buffer, a tris(hydroxymethyl)aminomethane (Tris) buffer, and sodium hydroxide. For certain of these embodiments, the elution reagent is phosphate buffer or Tris-EDTA buffer. For certain of these embodiments, releasing the captured nucleic acids is carried out at a pH of 7 to 10. This may also include a short and mild heating process.

For certain embodiments, including any one of the above embodiments of the method, the method further comprises detecting at least one of the nucleic acids. Known methods for detecting, which includes identifying, quantifying, or both, such as amplifying, hybridizing to a known probe, and sequencing, can be used. Examples of such methods are known, some of which are described in U.S. Pat. No. 6,617,105 (Rudi et al.) at column 9, line 21 through column 10, line 3.

Amplifying a nucleic acid may include, for example, producing a complementary polynucleotide of the nucleic acid or a portion of the nucleic acid in sufficient numbers for detection. Detection includes, for example, making an observation, such as detecting a fluorescence, which indicates the presence and/or amount of a polynucleotide. Amplifying the nucleic acid is carried out in the presence of certain reagents, depending on the amplifying method used. For example, useful reagents may include a nucleic acid amplifying enzyme, an oligonucleotide, a probe, nucleotide triphosphates, a buffer, and a salt. Additional reagents may be included to improve the amplifying process. Such reagents include, for example, a surfactant, a dye, a nucleic acid control, a reducing agent, Bovine Serum Albumin, dimethyl sulfoxide (DMSO), glycerol, ethylenediaminetetraacetic acid (EDTA), ethylene glycol-bis(2-aminoethylether)-N,N,N′,N′-tetraacetic acid (EGTA), and a combination thereof.

The nucleic acid amplifying enzyme can catalyze the production of a polynucleotide or a nucleic acid from an existing DNA or RNA template. Suitable nucleic acid amplifying enzymes include, for example, a DNA and/or RNA polymerase and a reverse transcriptase. Examples of the DNA polymerase include Taq DNA polymerase, Tfl DNA polymerase, Tth DNA polymerase, Tli DNA polymerase, and Pfu DNA polymerase. Examples of the RNA polymerase include T7 RNA polymerase. Examples of the reverse transcriptase include AMV reverse transcriptase, M-MLV reverse transcriptase, and M-MLV reverse transcriptase, RNase H minus Retroviral reverse transcriptase, such as M-MLV and AMV posses an RNA-directed DNA polymerase activity, a DNA directed polymerase activity, as well as an RNase H activity.

The “oligonucleotide” can be a primer, a terminating oligonucleotide, an extender oligonucleotide, or a promoter oligonucleotide. For certain embodiments, the oligonucleotide is a primer. Such oligonucleotides typically comprised of 15 to 30 nucleotide units, which determines the region (targeted sequence) of a nucleic acid to be amplified. Under appropriate conditions, the bases in the primer bind to complementary bases in the region of interest, and then the nucleic acid amplifying enzyme extends the primer as determined by the targeted sequence. A large number of primers are known and commercially available, and others can be designed and made using known methods.

Probes allow detection of amplification products (amplicons) by fluorescing, and thereby generating a detectable signal, the intensity of which is dependent upon the number of fluorescing probe molecules. Probe molecules can be comprised of an oligonucleotide and a fluorescing group coupled with a quenching group. Probes can fluoresce when separation or decoupling of the quenching group and the fluorescing group occurs upon binding to an amplicon or upon nucleic acid amplifying enzyme cleavage of the probe bound to the amplicon. Alternatively, a probe bound to the amplicon can fluoresce upon exposure to light of an appropriate wavelength. Examples of probes include TAQMAN probes (Applied Biosystems, Foster City, Calif.), molecular beacons, SCORPIONS probes (Eurogentec Ltd., Hampshire, UK), SYBR GREEN (Invitrogen, Carlsbad, Calif.), FRET hybridization probes (Roche Applied Sciences, Indianapolis, Ind.), Quantitect probes (Qiagen, Valencia, Calif.), and molecular torches.

The nucleotide triphosphates (NTPs), including ribonucleotide triphosphates and deoxyribonucleotides triphosphates as required, are used by the nucleic acid amplifying enzyme in the production of a polynucleotide or a nucleic acid from an existing DNA or RNA template. For example, when amplifying a DNA, a dNTP (deoxyribonucleotide triphosphate) set is used, which typically includes dATP (2′-deoxyadenosine 5′-triphosphate), dCTP (2′-deoxycytodine 5′-triphosphate), dGTP (2′-deoxyguanosine 5′-triphosphate), and dTTP (2′-deoxythimidine 5′-triphosphate).

Buffers are used to regulate the pH of the reaction media. A wide variety of buffers are known and commercially available. For example, morpholine buffers, such as 2-(N-morpholino)ethanesulfonic acid (MES), can be suitable for providing an effective pH range of about 5.0 to 6.5, imidazole buffers can be suitable for providing an effective pH range of about 6.2 to 7.8, and tris(hydroxymethyl)aminomethane (TRIS) buffers and certain piperazine buffers such as N-(2-hydroxyethyl)piperazine-N′-(2-ethanesulfonic acid) (HEPES) can be suitable for providing an effective pH range of about 7.0 to 9.0. The buffer can affect the activity and fidelity of nucleic acid amplifying enzymes, such as polymerases. For certain embodiments, the buffer is selected from at least one buffer which can regulate the pH in the range of 7.5 to 8.5. For certain of these embodiments, the buffer is a TRIS-based buffer. For certain of these embodiments, the buffer is selected from the group consisting of at least one of TRIS-EDTA, TRIS buffered saline, TRIS acetate-EDTA, and TRIS borate-EDTA. Other materials can be included with these buffers, such as surfactants and detergents, for example, CHAPS or a surfactant described below. The buffers may be free of RNase and DNase.

Salts can affect the activity of nucleic acid amplifying enzymes. For example, free magnesium ions are necessary for certain polymerases, such as Taq DNA polymerase, to be active. In another example, in the presence of manganese ions, Tfl DNA polymerase and Tth DNA polymerase can catalyze the polymerization of nucleotides into DNA, using RNA as a template. In a further example, the presence of certain salts, such as potassium chloride, can increase the activity of certain polymerases such as Taq DNA polymerase. Useful salts include magnesium, manganese, zinc, sodium, and potassium salts, for example, magnesium chloride, manganese chloride, zinc sulfate, zinc acetate, sodium chloride, and potassium chloride.

A surfactant can be included for enhancing lysing or de-clumping cells, improving mixing, and/or enhancing fluid flow, for example, in a device, such as a microfluidic device. The surfactant can be non-ionic, such as a poly(ethylene oxide)-poly(propylene oxide) copolymer available, for example, under the trade name PLURONIC, polyethylene glycol (PEG), polyoxyethylenesorbitan monolaurate available under the trade name TWEEN 20, 4-(1,1,3,3-tetramethylbutyl)phenyl-polyethylene glycol available under the trade name Triton X-100; anionic, such as lithium lauryl sulfate, N-lauroylsarcosine sodium salt, and sodium dodecyl sulfate; cationic, such as alkyl pyridinium and quaternary ammonium salts; zwitterionic, such as N—(C₁₀-C₁₆ alkyl)-N,N-dimethylglycine betaine (in the betaine family of surfactants); and/or a fluoro surfactant such as FLUORAD-FS 300 (3M, St. Paul, Minn.) and ZONYL (Dupont de Nemours Co., Wilmington, Del.).

A dye can be included in the reagent layer to impart a color or a fluorescence to the reagent layer or to a fluid which contacts the reagent layer. The color or fluorescence can provide visual evidence or a detectable light absorption or light emission evidencing that the reagent layer has been dissolved, dispersed, or suspended in the fluid which contacts the reagent layer. For certain embodiments, the dye is selected from the group consisting of fluorescent dyes, such as fluorescein, cyanine (which includes Cy3 and Cy5), Texas Red, ROX, FAM, JOE, SYBR Green, OliGreen, and HEX. In addition to these fluorescent dyes, ultraviolet/visible dyes, such as dichlorophenol, indophenol, saffranin, crystal violet, and commercially-available food coloring can also be used.

A nucleic acid control is a known amount of a nucleic acid or nucleic acid containing material dried-down with either the sample preparation or the amplification or detection reagents. This internal control can be used to monitor reagent integrity as well as inhibition from the sample material or specimen. Linearized plasmid DNA control is typically used as a nucleic acid internal control.

The reducing agent is a material capable of reducing disulfide bonds, for example in proteins which can be present in a sample material or specimen, and thereby reduce the viscosity and improve the flow and mixing characteristics of the sample material. For certain embodiments, the reducing agent preferably contains at least one thiol group. Examples of reducing agent include N-acetyl-L-cysteine, dithiothreitol, 2-mercaptoethanol, and 2-mercaptoethylamine.

Bovine Serum Albumin can be used to stabilize the enzyme during nucleic acid amplification; dimethyl sulfoxide (DMSO) can be used to inhibit the formation of secondary structures in the DNA template; glycerol can improve the amplification process, can be used as a preservative, and can stabilize enzymes such as polymerases; ethylenediaminetetraacectic acid (EDTA) and ethylene glycol-bis(2-aminoethylether)-N,N,N′N′-tetraacetic acid (EGTA) can be used as metal ion chelators and also to inactivate metal-binding enzymes (RNases) that may damage the reaction.

Nucleic acids can be detected by hybridizing the nucleic acid to a known probe. When the probe binds to a complementary region of the nucleic acid, the probe fluoresces as described supra. Sufficient amounts of nucleic acid and probe are needed for a measurable fluorescence.

A nucleic acid can also be detected by sequencing the nucleic acid. The use of very pure nucleic acid is desirable when using this method.

For certain embodiment, including any one of the above embodiments of the method comprising detecting, detecting includes processing the at least one nucleic acid by a step selected from the group consisting of amplifying the at least one nucleic acid, hybridizing the at least one nucleic acid, or a combination thereof.

For certain embodiments, including any one of the above embodiments of the method comprising separating support material with captured nucleic acids from the remaining material, the method further comprises labeling at least one of the nucleic acids.

For certain embodiments, including any one of the above embodiments of the method comprising releasing the captured nucleic acids from the support material, the method further comprising processing at least one of the nucleic acids in transforming a cell or in transfecting a cell.

For certain embodiments, including any one of the above embodiments of the method, the sample material is selected from the group consisting of a clinical sample, a food sample, and an environmental sample.

The sample material is any material which may contain microorganisms which can be lysed. The sample material can be a raw sample material or a processed sample material. Raw sample materials include, for example, clinical samples or specimens (blood, tissue, etc.), food samples (foods, feeds, raw materials for foods or feeds, etc.), environmental samples (water, soil, etc.), or the like. Processed sample materials include, for example, samples containing cells or viruses separated from a raw sample material. Some examples of sample material, such as clinical samples or specimens, include nasal, throat, sputum, blood, wound, groin, axilla, perineum, and fecal samples.

For certain embodiments, including any one of the above embodiments of the method, the sample material includes a plurality of cells, viruses, or a combination thereof. For certain of these embodiments, the sample material includes a plurality of cells. Cells can be prokaryotic or eukaryotic cells, and can include mammalian and non-mammalian animal cells, plant cells, algae, including blue-green algae, fungi, bacteria, protozoa, yeast, and the like. For certain of these embodiments, the cells are bacterial cells.

In another embodiment, there is provided a product comprising an array of spots on a carrier sheet, wherein at least a portion of the spots comprises a composition for processing a sample material, the composition comprising a nucleic acid binding support material comprising a substrate with functional groups attached to the substrate; a lysing enzyme; a water dispersible matrix material; and a saccharide selected from the group consisting of a monosaccharide, an oligosaccharide, and a combination thereof.

In another embodiment, there is provided a device for process a sample material, the device comprising at least one process chamber defining a volume for containing the sample material or a portion thereof; and a composition comprising a nucleic acid binding support material comprising a substrate with functional groups attached to the substrate; a lysing enzyme; a water dispersible matrix material; and a saccharide selected from the group consisting of a monosaccharide, an oligosaccharide, and a combination thereof; and wherein the composition is in the at least one process chamber.

In any of the above embodiments, the functional groups attached to the substrate of the nucleic acid binding support material include those which bind a nucleic acid. Suitable functional groups include, for example, a wobble sequence, a primary, secondary, tertiary, or quaternary ammonium group, and an immobilized-metal group. A wobble sequence can be attached to a substrate with oligo dT groups by tagging the end of a wobble sequence with oligo A. The oligo A tail binds to the oligo dT on the substrate. Substrates, such as microparticles, with oligo dT groups are commercially available. Alternatively, a wobble sequence can be biotinylated, and a substrate can have an avidin, such as streptavidin, attached thereto. The wobble sequence is attached to the substrate by the interaction between the biotin and avidin groups. The wobble sequence nonspecifically binds to nucleic acids. The bound nucleic acids can be released upon heating due to the denaturation of double stranded DNA.

Amino groups and groups containing amino groups can be attached to a substrate by a variety of known methods. Known methods can also be used to derivatize the amino groups to provide ammonium groups, such as —NR₃⁺ where R is H, C₁₋₄ alkyl, or a combination thereof. For example, chitosan can be bonded to a silica surface using a coupling agent such as 3-glycidoxypropyltrimethoxysilane. At a pH of 4-6, the amino groups of the chitosan are primary ammonium groups (—NH₃⁺), which nonspecifically bind to nucleic acids as described in International Publication No. WO 2006/088907. The nucleic acids can be released at an elevated pH, such as a pH of 8-10.

Immobilized-metal groups can formed by binding metal ions to acid groups, such as carboxylic and phosphonic acid groups. For certain embodiments, including any one of the above embodiments except those where the support material includes a wobble sequence or a ammonium group, the support material is preferably an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, a lanthanide or a combination thereof; y is an integer from 3 to 6; and x is 1 or 2. Although not wishing to be bound by theory, Applicants believe these metal ions bound to the support material interact with phosphate groups on the polynucleotides, causing the polynucleotides to bind to the immobilized-metal support material. Moreover, the captured polynucleotides can be released when desired with a short period of moderate heating and with a low concentration of a buffer which competes with or displaces the polynucleotide phosphate groups. The released polynucleotide in combination with the buffer can be used directly for downstream processes such as polynucleotide amplification.

The plurality of —C(O)O⁻ or —P(O)(—OH)₂₋ₓ(—O⁻)ₓ groups can be bound to the substrate in a number of ways. For example, the groups can be bound by covalent bonding, ionic bonding, hydrogen bonding, and/or van der Waals forces. The groups can be bound directly to the substrate, such as a substrate having a polymeric surface wherein a polymer has —C(O)O⁻ or —P(O)(—OH)₂₋ₓ(—O⁻)ₓ groups covalently bonded to the polymer chain. Polymers of this nature can include —C(O)OH or —P(O)(—OH)₂ substituted vinyl units, for example, acrylic acid, methacrylic acid, vinylphosphonic acid, and like units.

The —C(O)O⁻ or —P(O)(—OH)₂₋ₓ(—O⁻)ₓ groups can be bound indirectly to the substrate through a connecting group. For example, amino groups on a substrate can be contacted with a compound having multiple carboxy groups, such as nitrilotriacetic acid, to form an amide-containing connecting group which attaches one or more carboxy groups (two carboxy groups in the case of nitrilotriacetic acid) to the substrate. Substrates having available amino groups or which can be modified to have available amino groups are known to those skilled in the art and include, for example, agarose-based, latex-based, polystyrene-based, and silica-based substrates. Silica-based substrates such as glass or silica particles having —Si—OH groups can be treated with known aminosilane coupling agents, such as 3-aminopropyltrimethoxysilane, to provide available amino groups. Functional groups such as —C(O)OH or —P(O)(—OH)₂ can be attached to a substrate, for example, a substrate having a silica surface, using other known silane compounds.

The —C(O)O⁻ or —P(O)(—OH)₂₋ₓ(—O⁻)ₓ groups can also be bound indirectly to the substrate under conditions where these groups are attached to a molecule which binds to the substrate by electrostatic, hydrogen bonding, coordination bonding, van der Waals forces (hydrophobic interaction) or specific chemistry such as biotin-avidine interaction. For example, polymers bearing C(O)O⁻ or —P(O)(—OH)₂₋ₓ(—O⁻)ₓ groups can be coated on a surface with opposite charge using a Layer-by-Layer technique to build up a high density of polymer having C(O)O⁻ or —P(O)(—OH)₂₋ₓ(—O⁻)ₓ groups.

For a further example, monomers bearing C(O)O⁻ or —P(O)(—OH)₂₋ₓ(—O⁻)ₓ groups can be grafted to a polymer surface through plasma treatment.

Substrates having a plurality of carboxyl groups, e.g., —C(O)OH or —C(O)O⁻, are known and commercially available. For example, carboxylated microparticles are available under trade names such as DYNABEADS MYONE (Invitrogen, Carlsbad, Calif.) and SERA-MAG (Thermo Scientific, known as Seradyn, Indianapolis, Ind.).

The metal ions, M^{y+}, can be bound to acid groups by contacting the acid groups with an excess of metal ions, for example, as a solution of the metal salt, such as a nitrate salt. Other salts may be used as well, for example, chloride, perchlorate, sulfate, phosphate, acetate, acetylacetonate, bromide, fluoride, or iodide, salts.

The metal ion, M^{y+}, is chosen so that the metal ion can bind the phosphate portion of the polynucleotide sufficiently to bind the polynucleotide molecules present in a sample material. Moreover, the metal ion is also chosen to allow competitive binding with a metal-chelating reagent in a wash buffer to efficiently, preferably quantitatively, release or elute the polynucleotide molecules from the immobilized-metal support material at a low reagent concentration and under mild conditions. A low reagent concentration, which may include little or no addition of a salt to increase the ionic strength, can be about 0.1 M or less, 0.05 M or less, or 0.025 M or less. Mild conditions can include the low reagent concentration, a pH of about 7 to 10, a temperature of not more than about 95° C., preferably not more than about 65° C., or a combination thereof.

As indicated above, M can be zirconium, gallium, iron, aluminum, scandium, titanium, vanadium, yttrium, a lanthanide, or a combination thereof. A lanthanide includes any one of the lanthanide metals: lanthanum, cerium, praseodymium, promethium, samarium, europium, gadolinium, terbium, dysprosium, holmium, erbium, thulium, ytterbium, and lutetium. Lanthanum and cerium are preferred lanthanides. For certain of these embodiments, M is selected from the group consisting of zirconium, gallium, iron, aluminum, scandium, titanium, vanadium, lanthanum, and cerium.

For certain embodiments, including any one of the above methods, products, or devices, where the support material is an immobilized-metal support material, M is selected from the group consisting of zirconium, gallium, iron, and a combination thereof.

For certain embodiments, including any one of the above methods, products, or devices, where the support material is an immobilized-metal support material, y is 3 or 4.

For certain embodiments, including any one of the above methods, products, or devices, where the support material is an immobilized-metal support material, M^{y+} is Zr⁴⁺, Ga³⁺, Fe³⁺, or a combination thereof. For certain of these embodiments, M^{y+} is Zr⁴⁺ or Ga³⁺. For certain of these embodiments, M^{y+} is Zr⁴⁺.

For certain embodiments, including any one of the above methods, products, or devices, where the support material is an immobilized-metal support material, preferably the plurality of —C(O)O⁻ or —P(O)(—OH)₂₋ₓ(—O⁻)ₓ groups is a plurality of —C(O)O⁻ groups.

For certain embodiments, including any one of the above embodiments of the composition, method, product, or device, at least a portion of the composition is a dehydrated solid which includes the lysing enzyme, the water dispersible matrix material, and the saccharide. The dehydrated solid can be prepared by dispersing the lysis enzyme in an aqueous solution of the saccharide and water dispersible matrix material, dispensing the resulting mixture onto a surface, and drying the mixture to form a dry film in the form of a spot or other shape. The shape is controlled by the amount of mixture dispensed, the configuration in which the mixture is dispensed onto the surface, and/or the configuration and structure of the surface. For example, the dry film can be made thicker and/or to occupy a larger area by dispensing a larger amount of the composition. By dispensing the composition over a particular area, a desired shape can be made, for example, a circular area, an oval area, or any desired shape. Also, by configuring the surface with depressions or other microstructure of a particular shape, the dry film can be made into a particular shape. Alternatively, the dehydrated solid can be prepared by dispensing the mixture into liquid nitrogen to form frozen macrobeads, which are then lyophilized to provide dry macrobeads. Other forms of drying can be used, such as spray drying to form a powder. Tableting methods can be used to prepare dry tablets. Suitable methods exclude high shear and temperatures significantly above room temperature. The resulting dehydrated solid can be combined with the immobilized-metal support material. For example, the dehydrated solid can be place on or formed on a surface comprising the immobilized-metal support material. Such surfaces are those of the substrates, for example, a wall of a container, described infra.

For certain embodiments, including any one of the above embodiments wherein at least a portion of the composition is a dehydrated solid, preferably the dehydrated solid further includes the immobilized-metal support material. As indicated above, this composition has been found to be stable for an extended period of time, for example, for several months or more.

For certain embodiments, including any one of the above embodiments wherein at least a portion of the composition is a dehydrated solid, the dehydrated solid is a film, tablet, bead, or powder. For certain of these embodiments, the dehydrated solid is a film in the shape of a spot. For certain of these embodiments, the film is on a carrier sheet. For any of the above embodiments of the product, the dehydrated solid is a film, and each spot on the carrier sheet is the film. Suitable carrier sheets include, for example, glass, ceramic, and in some embodiments, preferably polymeric sheets, such as polyester, polyethylene, polypropylene, nylon, acylic, polycarbonate, and the like. Carrier sheets may also be membranes. The carrier sheet should be chosen so that the film adheres to the sheet sufficiently to allow handling, packaging, shipping, and the like, without the film releasing prematurely from the carrier sheet. For certain embodiments, the film can be readily removed from the carrier sheet for placement in a device. In certain alternative embodiments, a layer of adhesive is on the carrier sheet, and the film is on the adhesive layer. For such embodiments, an optional liner can be used to cover the adhesive side of the carrier sheet without damaging the film spots. The carrier sheet with the film spots can be made into a roll with the liner or with an appropriate choice of carrier sheet, optionally with a release coating on the side opposite the film spots. The carrier sheet itself can be a device with an array of wells defined in the sheet, at least a portion of the wells containing the dehydrated solid composition.

For certain embodiments where the composition is on a carrier sheet as a film, and an adhesive layer is between the carrier sheet and the film, the film can be placed in a device by placing the film, still on the adhesive layer and carrier sheet, in the device. This can be done by placing the film with adhesive layer and carrier sheet in a process chamber of the device so that a sample material can contact the film. In one example, the film with adhesive layer and carrier sheet act as a window of the process chamber, with the film facing within the chamber and the adhesive sealing the carrier sheet to the chamber.

For certain embodiments, including any one of the above embodiments wherein at least a portion of the composition is a dehydrated solid, except were the dehydrated solid is a film, the dehydrated solid is a bead or tablet. Beads or tablets can be conveniently used in pick-and-place methods for placing the composition in a device.

For certain embodiments, including any one of the above compositions, methods, or devices, except those where at least a portion of the composition is a dehydrated solid, the composition further comprises an aqueous liquid carrier, wherein the support material, the lysing enzyme, the water dispersible matrix material, and the saccharide are dispersed in the aqueous liquid carrier. Suitable aqueous liquid carriers include, for example, an aqueous buffer at a pH of about 4.5 to about 6.5. In one example, the binding buffer is MES (4-morpholineethanesulfonic acid) at about 0.2 M to about 0.1 M and at a pH of about 5.5. A non-ionic surfactant such as PLURONIC L64 (a polyoxyethylene-polyoxypropylene block copolymer available from BASF (Mt. Olive, N.J.) or TRITON series, including TRITON X-100 (polyoxyethylene(10) isooctylphenyl ether available from Sigma-Aldrich, St. Louis, Mo.), TWEEN series, BRIJ series, or NP-40 can be included for improved flow and mixing. Surfactants may also reduce or prevent clumping of bacterial cells. Other buffers which can be similarly used include succinic acid, acetate, or citrate.

Compositions including the aqueous liquid carrier can be printed. This is unexpected, since the support material, an example of which includes magnetic particle substrates, could separate out and also clog the printer. Accordingly, for certain embodiments, including any one of the above compositions which has an aqueous liquid carrier, the composition is printable.

For certain embodiments, including any one of the above embodiments except where the composition includes an aqueous carrier or where the dehydrated solid includes the support material, the substrate (of the solid support material) is selected from the group consisting of a gel, a film, a sheet, a membrane, a particle, a fiber, a strip, a tube, a column, a well, a wall of a container, and a combination thereof.

For certain embodiments, including any one of the above embodiments, the substrate (of the solid support material) is particles. The particles can be microparticles, which include microspheres, microbeads, and the like. Such particles can be resin particles, for example, agarose, latex, polystyrene, nylon, polyacylamide, cellulose, polysaccharide, or a combination thereof, or inorganic particles, for example, silica, aluminum oxide, or a combination thereof. Such particles can be magnetic or non-magnetic. Such particles can have a diameter of about 0.01 microns to about 10 microns. For certain of these embodiments, the particles are magnetic particles. For certain of these embodiments, the magnetic particles have a diameter of about 0.02 microns to about 5 microns.

For certain embodiments, including any one of the compositions, methods, products, or devices, the lysing enzyme is selected from the group consisting of lysostaphin, lysozyme, mutanolysin, a proteinase, a pronase, a cellulase, cell wall peptidoglycan degrading enzyme, and a combination thereof. For certain of these embodiments, the lysing enzyme is lysostaphin or lysozyme.

In any of the above methods where microorganisms are lysed, the lysing can include methods in addition to lysing with the enzyme(s). For example, chemical lysing can be carried out using a surfactant, alkali, heat, or other means. When alkali is used for lysis, a neutralization reagent may be used to neutralize the solution or mixture after lysis. Mechanical lysis can be accomplished by mixing or shearing using solid particles or microparticles such as beads or microbeads. Sonication may also be used for lysis. A surfactant or detergent such as sodium dodecylsulfate (SDS), lithium laurylsulfate (LLS), TRITON series, TWEEN series, BRIJ series, NP series, CHAPS, N-methyl-N-(1-oxododecyl)glycine, or the like, buffered as needed can be used. A chaotrope such as guanidium hydrochloride, guanidium thiacyanate, sodium iodide, or the like can be used.

For certain embodiment, including any one of the above methods, the method is carried out within a microfluidic device.

As indicated above, the compositions described herein include a water dispersible matrix material. Suitable matrix materials are compatible with the lysis enzyme and the support material and include carbohydrates and water soluble polymers. As used herein “water dispersible” and “water soluble” means that the material can be dissolved, dispersed, or suspended in water at a temperature that is at least room temperature and preferably not more than about 75° C. After an aqueous dispersion of the matrix material, lysis enzyme, and the support material is dried, the matrix material can hold or contain the lysis enzyme and the support material. The matrix material can also increase adhesion of the dried composition to a carrier sheet and allow the dried composition to be coated in a wider range of thicknesses than would otherwise be possible. The ability to prepare the dried composition in a wide range of thicknesses allows a wider range of lysis enzyme, support material, and/or other reagent amounts to be provided. The matrix material should also be dispersible in water or an aqueous buffer within a short period of time to allow a quick resuspension of the lysis enzyme and the support material if included with the dried matrix material. The matrix material may be used at levels of at least about 1 or about 2 percent by weight of the composition. The matrix material may be used a levels of not more than about 10 or about 8 percent by weight.

For certain embodiments, including any one of the above compositions, methods, products, or devices, the water dispersible matrix material is selected from the group consisting of a dextran, a dextrin, alginic acid and salts thereof, a glucan, pullulan, glycogen, ficoll, pectin, chitosan, xylan, a carrageenan, guar gum, locust gum, gum arabic, xanthan gum, acacia gum, hydroxyethylcellulose, hydroxypropylcellulose, hydroxypropylmethylcellulose, carboxymethylcellulose and salts thereof, poly(vinyl alcohol), polyvinylpyrrolidone, and a combination thereof. The poly(vinyl alcohol) is preferably at least 80% hydrolyzed or at least 90% hydrolyzed and has a weight average molecular weight of about 30,000 to about 70,000. For certain of these embodiments, the water dispersible matrix material is dextran. For certain of these embodiments, the dextran has a molecular weight of 5,000 to 60,000.

A saccharide is also included in the compositions described herein. The saccharide stabilizes the lysis enzyme during drying, so that the enzyme is not deactivated when the composition is dried. Additionally, the saccharide shortens the time required to resuspend the lysis enzyme, the support material, the matrix material, and other components that may be present in the dried composition. The saccharide should be water soluble, dissolving quickly in water or an aqueous buffer, preferably at room temperature. The saccharide may be used at levels of at least about 2 or about 5 percent by weight of the composition. The saccharide may be used a levels of not more than about 20 or about 15 percent by weight. For certain embodiments, including any one of the above compositions, methods, products, or devices, the saccharide is a monosaccharide or a disaccharide. For certain of these embodiments, the saccharide is disaccharide. For certain of these embodiments, the saccharide is sucrose, trehalose, or a combination thereof.

For those embodiments where the substrate for the support material is particles and an aqueous liquid carrier is included in the composition, the water dispersible matrix material and the saccharide dispersed in the aqueous liquid carrier has been found to provide a media that stabilizes the particle suspension without visible settling over a period of at least several hours. This allows these compositions to be reproducibly printed, such that a given printed spot contains essentially the same amount of support material as a later printed spot.

The device for processing sample material described above can provide a location or locations for using the above described compositions and carrying out the above described methods. The device can provide conditions not only for sample preparation, including lysing microorganisms and capturing nucleic acids, but also for other processes such as nucleic acid amplification, and/or detection. The sample material may be located in one or a plurality of chambers. The device may provide uniform and accurate temperature control of one or more chambers included in the device. The device may provide channels between chambers, for example, such that sample preparation may take place in one or more chambers, and nucleic acid amplification and detection may take place in one or more other chambers. For certain embodiments, including any one of the above embodiments which include the device for processing sample material, the device for processing sample material is a microfluidic device. Some examples of microfluidic devices are described in U.S. Publication Numbers 2002/0064885 (Bedingham et al.); US2002/0048533 (Bedingham et al.); US2002/0047003 (Bedingham et al.); and US2003/138779 (Parthasarathy et al.); U.S. Pat. Nos. 6,627,159; 6,720,187; 6,734,401; 6,814,935; 6,987,253; 7,026,168, and 7,164,107; and International Publication No. WO 2005/061084 A1 (Bedingham et al.).

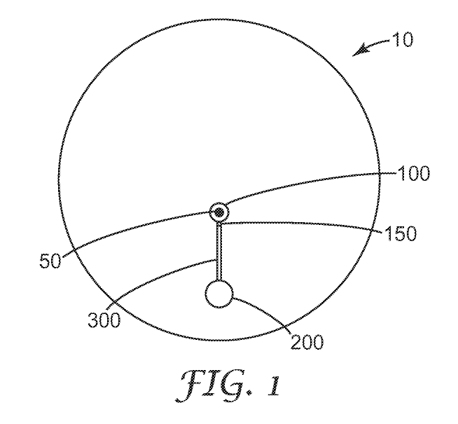

One illustrative device for processing sample material is the microfluidic device depicted in FIG. 1. The device 10 can be in the shape of a circular disc as illustrated in FIG. 1, although other shapes can be used. Preferred shapes are those that can be rotated. The device 10 of FIG. 1 comprises a first chamber 100 and a second chamber 200 which can be in fluid communication with the first chamber 100 via channel 300. The shape of chambers 100 and 200 can be circular as illustrated in FIG. 1, although other shapes, for example, oval, tear-drop, triangular, and many others can be used. FIG. 1 illustrates one combination of chamber 100 and chamber 200, but it is to be understood that a plurality of such combinations can be included in device 10 and may be desirable for simultaneously processing a plurality of samples.

The device 10 illustrated in FIG. 1 includes the composition for processing a sample material 50 in chamber 100. The composition for processing a sample material 50 can include the support material (magnetic or non-magnetic particles such as microparticles (microspheres, microbeads, etc.), resin particles, or the like). In another alternative, the support material can be an interior wall of chamber 100.

Sample preparation such as binding cells or viruses, lysing, digesting debris from cells or viruses, polynucleotide binding, washing, and the like can be carried out in chamber 100 prior to moving material in chamber 100 through channel 300 and into chamber 200. After a polynucleotide has been separated from the sample material by binding the polynucleotide from the lysed microorganisms to the support material, the support material can be moved to chamber 200, or the polynucleotide can be eluted from the support material and the resulting eluant moved to chamber 200. The channel 300 can provide a path for a fluid and/or the support material in chamber 100 to move into chamber 200. This can be carried out, for example, by applying a sufficient g-force to the fluid and/or the support material in the form of particles to force the material through channel 300 and into chamber 200. Alternatively, a pressure differential can be applied to channel 300, for example, by reducing the pressure in chamber 200, by increasing the pressure in chamber 100, or both, thereby causing material in chamber 100 to move through channel 300 and into chamber 200. Chamber 100 or channel 300 can be equipped with optional valve 150. Valve 150 can be fabricated to open by exposure to a sufficient g-force, by melting, by vaporizing, or the like. For example, the valve can be fabricated in the form of a septum in which an opening can be formed through laser ablation, focused optical heating, or similar means. Such valves are described, for example in U.S. Patent Application Publication Nos. 2005/0126312 A1 (Bedingham et al.) and 2005/0142571 A1 (Parthasarathy et al.).

Although not shown in FIG. 1, chambers 100 and 200 and channel 300 can be in fluid communication with other chambers, channels, reservoirs, and/or the like. These can be used to facilitate supplying or removing various reagents, sample material(s), or a component(s) of a sample material to or from chambers 100 or 200 as needed. For example, sample materials, compositions described herein with lysing enzyme, digestion reagents, wash buffers, binding buffers, elution buffers, and/or the like can be supplied to and/or removed from chamber 100, and primers, nucleotide triphosphates, amplifying enzymes, probes, buffers, and/or the like can be supplied to chamber 200. Individual reagents or combinations of reagents can be placed in different chambers, whether included in the device 10 or in any embodiment of the device described herein, to subsequently contact the reagents with the sample material or a component of the sample material as desired.

For certain embodiments, including any one of the above embodiments of the device for processing sample material, at least one chamber of the device includes at least one additional reagent which can be used in at least one step of a nucleic acid manipulation technique. For certain of these embodiments, the at least one additional reagent can be used in a step of sample preparation, a step of nucleic acid amplification, and/or a step of detection in a process for detecting or assaying a nucleic acid. Sample preparation may include, for example, lysing a biological material containing a nucleic acid, for example, cells or viruses, digesting cellular debris, isolating at least one polynucleotide or nucleic acid from a biological sample, and eluting a nucleic acid. Nucleic acid amplification may include, for example, producing a complementary polynucleotide of a polynucleotide or a portion of a polynucleotide in sufficient numbers for detection. Detection includes, for example, making an observation, such as detecting a fluorescence, which indicates the presence and/or amount of a polynucleotide. For certain of these embodiments, at least one chamber of the device includes at least one additional reagent selected from the group consisting of a nucleic acid amplifying enzyme, an oligonucleotide, a probe, nucleotide triphosphates, a buffer, a salt, a surfactant, a dye, a nucleic acid control, a reducing agent, Bovine Serum Albumin, dimethyl sulfoxide (DMSO), glycerol, ethylenediaminetetraacetic acid (EDTA), ethylene glycol-bis(2-aminoethylether)-N,N,N′,N′-tetraacetic acid (EGTA), and a combination thereof. For certain of these embodiments, at least one chamber of the device includes at least one additional reagent selected from the group consisting of a nucleic acid amplifying enzyme, an oligonucleotide, a probe, nucleotide triphosphates, a buffer, and a salt.

Objects and advantages of this invention are further illustrated by the following examples, but the particular materials and amounts thereof recited in these examples, as well as other conditions and details, should not be construed to unduly limit this invention.

### EXAMPLES

### Preparative Example 1

### Preparation of Metal-Ion Mediated Magnetic Microparticles

Metal-ion mediated magnetic microparticles, for use as an immobilized-metal support material, were prepared from magnetic particles with surface carboxylic acid groups and with a diameter of about 1 micron (SERA-MAG Magnetic Particles from Thermo Scientific, known as Seradyn, Indianapolis, Ind.). The carboxylated magnetic microparticles were placed in a tube and washed by attracting them to the wall of the tube using a magnet, removing the liquid by aspiration, replacing the liquid volume with the wash solution, removing the tube from the magnetic field, and agitating the tube to resuspend the microparticles.

Prior to metal-ion treatment, the magnetic microparticles were washed twice with 0.1 M MES buffer, pH 5.5 (containing 0.1% TRITON X-100) and then re-suspended in the same buffer. Following the wash step, 0.2 mL of 0.1 M gallium (III) nitrate, or ferric nitrate or zirconium (IV) nitrate in 0.01 M HCl solution per milligram of magnetic microparticles was added to the magnetic microparticle suspension. The mixture was allowed to shake gently for 1 h at room temperature and subsequently washed with the above MES buffer to remove excess metal ions. The resulting metal-ion mediated magnetic microparticles (Ga(III)-microparticles, Fe(III)-microparticles, and Zr(IV)-microparticles) were resuspended and stored at 4° C. in MES buffer.

### Example 1

### Dried Films Containing Metal-Ion Mediated Magnetic Microparticles with Various Levels of Dextran

Three compositions were prepared, each containing 100 μg Ga(III)-microparticles/6 μL of volume, 10 weight percent sucrose, and 1, 2, and 5 weight percent, respectively, dextran (average molecular weight of 30,000-40,000) in TE buffer (10 mM Tris and 1 mM EDTA). Each of these was applied to the surface of a polyester (PET) film by dispensing 6 μL volumes as spots onto the PET. After vacuum drying overnight at room temperature, each of the spots was a dried film. Each of the dried films was peeled off of the PET. The 5 weight percent dextran was not only peeled off easily, but this amount of dextran provided the best quality dried film of those tested, with the most uniform appearance. Each of the dried films was resuspended in water. The 5 weight percent dextran film resuspended within 1 minute, while the others took somewhat longer.

### Comparative Example 1

Example 1 was repeated except that no dextran was included in the composition. After vacuum drying, the dried films were very fragile and easy to crack when they were transferred from the PET. Resuspending these dried films in water took place much more slowly than the dried films containing dextran.

### Example 2

### Dried Microbeads Containing Metal-Ion Mediated Magnetic Microparticles with Various Levels of Dextran

Three compositions were prepared, each containing 100 μg Ga(III)-microparticles/12.5 μL of volume, 10 weight percent sucrose, and 1, 2, and 5 weight percent, respectively, dextran (average molecular weight of 30,000-40,000) in 0.2 M MES buffer. Each of these was frozen as small droplets in liquid nitrogen by dispensing 12.5 μL volumes onto the liquid nitrogen and kept there for 5 minutes. The resulting macrobeads, which had a diameter of about 1 mm, were then lyophilized overnight to dryness. The dried macrobeads with 5 weight percent dextran appeared the most uniform and best quality of those evaluated, and were easily transferred without evidence of breakage. At 1 weight percent dextran, the dried macrobeads were more fragile. Each of the dried macrobeads was resuspended in water and found to be resolubilized in less than 30 seconds. The macrobeads were observed to be porous, which speeded resolubilization.

### Example 3

### Effects of Dextran and Sucrose on DNA Binding and Nucleic Acid Amplification

In this experiment, 100 μg of Ga(III)-microparticles are used to bind 10⁵ cfu equivalent MRSA DNA (about 1.8 ng) in TEP buffer (10 mM Tris, 1 mM EDTA, and 0.2 weight percent PLURONIC L64 (BASF, Mt. Olive, N.J.)) with 10 weight percent sucrose and 1, 2, and 5 weight percent dextran. Samples are made by spiking MRSA DNA (˜10⁵ cfu MRSA) into 100 μL TEP, and then the macrobeads from Example 3 containing 1, 2, and 5 weight percent dextran are added to each sample, respectively, one macrobead in each sample. Additional mixtures are prepared as above, except that instead of adding macrobeads, 100 μg of Ga(III)-microparticles are added to each sample, sucrose is added to each sample at 10 weight percent, and dextran is added to the samples at 0, 1, 2, and 5 weight percent, respectively. Further additional mixtures are made but with no sucrose and no dextran. The resulting mixtures are incubated for 10 minutes at room temperature. After magnetically removing the supernatant, the Ga(III)-microparticles are washed twice with 100 μL TEP. The Ga(III)-microparticles are resuspended in 100 μL 20 mM sodium phosphate buffer (pH 8.5, 0.2 weight percent PLURONIC L64). The resulting suspensions are assayed for released MRSA DNA using 5 μL of each suspension for mecA-FAM RT-PCR analysis.

Five microliters of each sample are subjected to real-time PCR amplification for mecA gene using the following optimized concentrations of primers, probe and enzyme, as well as thermo cycles. The sequence of all primers and probes listed below are given in the 5′→3′ orientation and are known and described in Francois, P., et al., Journal of Clinical Microbiology, 2003, volume 41, 254-260. The forward mecA primer is CATTGATCGCAACGTTCAATTT (SEQ ID NO. 1). The mecA reverse primer is TGGTCTTTCTGCATTCCTGGA (SEQ ID NO. 2). The mecA probe sequence, TGGAAGTTAGATTGGGATCATAGCGTCAT (SEQ ID NO. 3), wis dual labeled by 6-carboxyfluorescein (FAM) and IBFQ (IOWA BLACK FQ, Integrated DNA Technologies, Corniville, Iowa) at 5′- and 3′-position, respectively. PCR amplification is performed in a total volume of 10 μL containing 5 μL of sample and 5 μL of the following mixture: two primers (0.5 μL of 10 μM of each), probe (1 μL of 2 μM), MgCl₂ (2 μL of 25 mM) and LightCycler DNA Master Hybridization Probes (1 μL of 10×, Roche, Indianapolis, Ind.). Amplification is performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

DNA control samples consisting of DNA (equivalent to the amount used in the binding experiments) suspended in the phosphate buffer are assayed at the same time. The control DNA samples are not reacted with metal-ion mediated microparticles.

Table 1 shows representative results which can be obtained from mecA PCR assay.

TABLE 1
PCR Assay Data

| Sample | Cₜ | |
|---|---|---|
| Ga(III)-microparticles, 10% sucrose, and 5% | 24 ± 1 | 24 ± 1 |
| dextran from macrobeads | | |
| Ga(III)-microparticles, 10% sucrose, and 2% | 24 ± 1 | 24 ± 1 |
| dextran from macrobeads | | |
| Ga(III)-microparticles, 10% sucrose, and 1% | 24 ± 1 | 24 ± 1 |
| dextran from macrobeads | | |
| Ga(III)-microparticles, 10% sucrose, and 5% | 24 ± 1 | 24 ± 1 |
| dextran directly added | | |
| Ga(III)-microparticles, 10% sucrose, and 2% | 24 ± 1 | 24 ± 1 |
| dextran directly added | | |
| Ga(III)-microparticles and 10% sucrose directly | 24 ± 1 | 24 ± 1 |
| added | | |
| Ga(III)-microparticles directly added, no sucrose | 24 ± 1 | 24 ± 1 |
| or dextran | | |
| DNA Control | 24 ± 1 | 24 ± 1 |

(The sample is suspended in 100 μL of buffer and 5 μL of the resulting sample and 5 μL of PCR Master mixture is used for PCR amplification.)
Ct values are reported from duplicate PCR reactions for each sample.

The similar Ct values for all samples indicates no effect of sucrose or dextran on DNA binding to the microparticles or on nucleic acid amplification or any part of the PCR assay.

### Example 4

### Stability of Dried Films and Macrobeads Containing Lysostaphin and Ga(III)-Microparticles

A solution is prepared by combining dextran (0.5 g), sucrose (1.0 g), sodium azide (10 mg), and 10 mL of 0.2 mM MES (pH 5.5, containing 0.1 weight percent TRITON X-100). Lysostaphin (Sigma, St Lous, Mo.) (0.5 mg) is added to 0.74 mL of this solution. The resulting solution has a lysostaphin concentration of 675 μg/mL. Ga(III)-microparticles (2 mg, isolated from 200 μL of 10 mg/mL Ga(III)-microparticles in 0.1 M MES from Preparative Example 1) are resuspended in 95 μL of the lysostaphin solution. Portions of the resulting suspension are dispensed into liquid nitrogen by pipetting 11 μL volumes of the suspension into liquid nitrogen to form frozen macrobeads. The frozen macrobeads are lyophilized overnight to provide dried macrobeads, which are stored in sealed tubes at ambient conditions. Portions of the above suspension are also made into dried films by dispensing 11 μL volumes of the suspension onto a PET film and vacuum drying the resulting spots overnight. The resulting dried film spots are peeled off of the PET and transferred to a tube, which is sealed and stored at ambient conditions.

After storage for various periods of time, the dried films and macrobeads are evaluated for their ability to lyse MRSA and capture the MRSA DNA. MRSA 240 (ATTC BAA-4) is serially diluted from stock (1.5×10⁹ cfu/mL) by a factor of 10 in TEP for each dilution. The third (E-3), fourth (E-4), and/or fifth (E-5) MRSA dilutions are tested. To 183 μL of TEP, 67 μL of MRSA dilution is added followed by addition of a dried film or dried macrobead to provide a suspension. For controls, MRSA dilutions (67 μL) are added to mixtures of lysostaphin (6 μg) and Ga(III)-microparticles (100 μg isolated from 10 mg/mL Ga(III)-microparticles in 0.1 M MES from Preparative Example 1) in a total volume of 250 μL TEP to provide a control suspension. All suspensions are gently shaken for 10 minutes at room temperature. The microparticles in each suspension are separated and washed twice with 100 μL TEP. The microparticles are resuspended in 100 μL of 20 mM sodium phosphate buffer (pH 8.5, 0.2% PLURONIC L64) and heated for 5 minutes in a water bath at 95° C. The supernatant from each suspension is isolated, and 5 μL of each supernatant is used for mecA PCR assay as described in Example 3. Representative results can be obtained as shown in Table 2 below.

TABLE 2
Lysis and Nucleic Acid Binding Stability of Dried Films and Macrobeads
Containing Lysostaphin and Nucleic Acid Binding Support Material

| | After 1 Day | | After 6 Days | | After 45 Days | | After 185 Days | |
|---|---|---|---|---|---|---|---|---|
| Sample | E-4 | E-5 | E-4 | E-5 | E-4 | E-5 | E-3 | E-4 |
| Dried Film | 23 ± 1 | 26 ± 1 | 23 ± 1 | 26 ± 1 | 23 ± 1 | 26 ± 1 | NA | NA |
| Dried | 23 ± 1 | 26 ± 1 | 23 ± 1 | 26 ± 1 | 23 ± 1 | 26 ± 1 | 20 ± 1 | 23 ± 1 |
| Macrobeads | | | | | | | | |
| Control | 23 ± 1 | 26 ± 1 | 23 ± 1 | 26 ± 1 | 23 ± 1 | 26 ± 1 | 20 ± 1 | 23 ± 1 |

The results in Table 2 show that the dried films and macrobeads were very stable at ambient conditions. Stability without degradation for more than six months is shown.

### Example 5

### Printing Compositions Containing Magnetic Microparticles

A 10 mL volume of solution containing 10 weight percent sucrose and 5 weight percent dextran in 0.2 M MES buffer (pH 5.5 with 0.1 weight percent TRITON X-100) was mixed with Brilliant Bromcresol Blue dye (5 mg) and 1 micron Ga(III)-microparticles from Preparative Example 1 (50 mg/mL of solution) to provide a composition for printing. The composition was printed using a BIODOT AD3200 BIOJET PLUS printer (Biodot Inc. Irvine, Calif. 92614). An array of spots was printed at 2 μL/spot on the adhesive side of a PET film coated with a tackified silicone-polyurea polymer adhesive, each spot with a diameter of about 2 mm and containing about 100 μg of magnetic microparticles. Additional spots were printed on the side of the PET film with no adhesive. In this case the diameter of the spots was about 3 mm. The spots were air dried overnight. All spots were uniform in size with no observable cracks or flaws.

### Example 6

### Uniformity of Printed Compositions Containing Magnetic Microparticles

A composition for printing was prepared as in Example 6, except that all amounts were increase by 10 fold. Four PET sheets were printed with 800 spots each over a period of 4 hours as described in Example 6. After drying overnight in air, five consecutive spots were collected from five different rows of spots on each sheet, including the first five spots and the last five spots printed on each sheet. All spots were weighed, and the results are shown in Table 3.

Even though no lysing enzyme was included in Examples 5 and 6, because the lysing enzyme essentially dissolves in the buffer, it would not change the results of these Examples if included.

TABLE 3
Uniformity of Printed Dried Film Spots

| | | Average | | Average | |
|---|---|---|---|---|---|
| | Group | Weight | | Weight | |
| | of 5 | of Each | | of Spots on | Standard |
| Sheet | Spots | Spot (mg) | CV % | Sheet (mg) | Deviation |
| 1 | 1 | 1.85 | 3.46 | 1.86 | 0.06 |
| | 2 | 1.96 | | | |
| | 3 | 1.86 | | | |
| | 4 | 1.85 | | | |
| | 5 | 1.78 | | | |
| 2 | 1 | 1.60 | 6.96 | 1.77 | 0.12 |
| | 2 | 1.70 | | | |
| | 3 | 1.90 | | | |
| | 4 | 1.87 | | | |
| | 5 | 1.76 | | | |
| 3 | 1 | 1.63 | 4.86 | 1.73 | 0.08 |
| | 2 | 1.70 | | | |
| | 3 | 1.75 | | | |
| | 4 | 1.86 | | | |
| | 5 | 1.72 | | | |
| 4 | 1 | 1.60 | 6.47 | 1.84 | 0.12 |
| | 2 | 1.87 | | | |
| | 3 | 1.90 | | | |
| | 4 | 1.89 | | | |
| | 5 | 1.92 | | | |
| | 6 | 1.84 | | | |

| | |
|---|---|
| Overall Ave. | 1.80 |
| Overall | 0.11 |
| Standard Dev. | |
| Overall CV % | 6.11 |

Group 1 and group 5 from each sheet is the first five spots and the last five spots, respectively, from each sheet.

All references and publications or portions thereof cited herein are expressly incorporated herein by reference in their entirety into this disclosure. Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

1. A composition for processing a sample material, the composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof.

2. The composition of claim 1, wherein the support material is an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups;
wherein M is selected from the group consisting of zirconium, gallium, iron, aluminum, scandium, titanium, vanadium, yttrium, a lanthanide or a combination thereof; y is an integer from 3 to 6; and x is 1 or 2.

3. The composition of claim 1, wherein at least a portion of the composition is a dehydrated solid which includes the lysing enzyme, the water dispersible matrix material, and the saccharide.

4. The composition of claim 3, wherein the dehydrated solid further includes the support material.

5. The composition of claim 3, wherein the dehydrated solid is a film, tablet, bead, or powder.

6-8. (canceled)

9. The composition of claim 1, further comprising an aqueous liquid carrier, wherein the support material, the lysing enzyme, the water dispersible matrix material, and the saccharide are dispersed in the aqueous liquid carrier, and wherein the composition is printable.

10-13. (canceled)

14. The composition of claim 2, wherein M is selected from the group consisting of zirconium, gallium, iron, and a combination thereof.

15-16. (canceled)

17. The composition of claim 2, wherein the plurality of —C(O)O⁻ or —P(O)(—OH)₂₋ₓ(—O⁻)ₓ groups is a plurality of —C(O)O⁻ groups.

18. The composition of claim 1, wherein the lysing enzyme is selected from the group consisting of lysostaphin, lysozyme, mutanolysin, a proteinase, a pronase, a cellulase, cell wall peptidoglycan degrading enzyme, and a combination thereof.

19. (canceled)

20. The composition of claim 1, wherein the water dispersible matrix material is selected from the group consisting of a dextran, a dextrin, alginic acid and salts thereof, a glucan, pullulan, glycogen, ficoll, pectin, chitosan, xylan, a carrageenan, guar gum, locust gum, gum arabic, xanthan gum, acacia gum, hydroxyethylcellulose, hydroxypropylcellulose, hydroxypropylmethylcellulose, carboxymethylcellulose and salts thereof, poly(vinyl alcohol), polyvinylpyrrolidone, and a combination thereof.

21-22. (canceled)

23. The composition of claim 1, wherein the saccharide is sucrose, trehalose, or a combination thereof.

24. A method of processing a sample material comprising:
providing a composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof;
providing a sample material suspected of having a plurality of microorganisms which can be lysed;
contacting the sample material with the composition;
wherein as least a portion of the microorganisms are lysed, the lysed microorganisms release nucleic acids, and at least a portion of the released nucleic acids are captured by the support material; and
separating the support material with captured nucleic acids from any remaining materials.

25. (canceled)

26. The method of claim 24, further comprising releasing the captured nucleic acids from the support material.

27. The method of claim 24, further comprising detecting at least one of the nucleic acids.

28-30. (canceled)

31. The method of claim 24, wherein the sample material is selected from the group consisting of a clinical sample, a food sample, and an environmental sample.

32. A product comprising an array of spots on a carrier sheet, wherein each of the spots comprises a composition for processing a sample material, the composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof.

33. A device for process a sample material, the device comprising:
at least one process chamber defining a volume for containing the sample material or a portion thereof; and
a composition comprising:
a nucleic acid binding support material comprising a substrate with functional groups attached to the substrate;
a lysing enzyme;
a water dispersible matrix material; and
a saccharide selected from the group consisting of a monosaccharide, an oligosaccharide, and a combination thereof;
wherein the composition is in the at least one process chamber.

34. The method of claim 24, wherein the support material is an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, a lanthanide or a combination thereof; y is an integer from 3 to 6; and x is 1 or 2.

35-56. (canceled)

57. The product of claim 32, wherein the support material is an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, a lanthanide or a combination thereof; y is an integer from 3 to 6; and x is 1 or 2.

58. The device of claim 33, wherein the support material is an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, a lanthanide or a combination thereof; y is an integer from 3 to 6; and x is 1 or 2.
